(19) United States
(12) Patent Application Publication
(10) Pub. No.: US 2016/0220481 A1
(43) Pub. Date: Aug. 4, 2016

(54) Low-Sodium Effervescent Pharmaceutical Formulations

(71) Applicant: EFFRX PHARMACEUTICALS SA, Freienbach (CH)

(72) Inventors: TIMO SCHMIDT, SCHINDELLEGI (CH); MARSHALL A. HAYWARD, Bridgewater, NJ (US)

(73) Assignee: EFFRX PHARMACEUTICALS SA, FREIENBACH (CH)

(21) Appl. No.: 15/021,214

(22) PCT Filed: Mar. 1, 2014
(86) PCT No.: PCT/IB2014/000826

§ 371 Date: Mar. 10, 2016

Related U.S. Application Data
(60) Provisional application No. 61/880,875, filed on Sep. 21, 2013.

(51) Int. Cl. A61K 9/46; A61K 31/7042; A61J 3/10; A61K 31/403; A61K 9/20; A61K 31/155; A61K 31/4985

(52) U.S. Cl. CPC A61K 9/0007; A61K 31/155; A61K 31/7042; A61K 31/4985; A61K 31/403; A61K 9/2013; A61K 9/2009; A61K 9/2095; A61J 3/10

(57) ABSTRACT

A stable effervescent tablet granule or powder composition free from excipients that may react with an effervescing organic acid component, comprising: an effective amount of a antidiabetic drug, an effervescing organic acid component, an effervescing base component; buffers, to modulate gastric ρïí after ingestion, essentially free of solium; wherein said composition is completely solubilised within 5 minutes without stirring in 3 to 8 fluid ounces of water at between 5-20° C.

### FIELD OF THE INVENTION

Effervescent formulations for treating type-2 diabetes and other chronic diseases based on potassium carbonates or potassium bicarbonates.

### BACKGROUND OF THE INVENTION

Type 2 diabetes is a chronic, progressive disease associated with high morbidity and mortality, it is characterized by peripheral insulin resistance, impaired regulation a hepatic glucose production, and declining β-cell function in the pancreas. These events eventually lead to β-cell failure and a reduction in insulin secretion. Complication of insulin resistance in diabetes and pre-diabetes and co-morbitities are also important considerations, as exemplified by such disorders as polycystic ovarian syndrome (PCOS).

Diet and exercise form the basis of all initial programs for treating patients with type 2 diabetes. If blood glucose levels remain elevated despite dietary control and exercise, treatment with an oral medication is usually recommended. Current oral therapeutic agents for type 2 diabetes mellitus include sulfonylureas, biguanides, thiazolidinediones, α-glucosidase inhibitors, and insulin.

Sulfonylureas were the first oral antidiabetic agents and they continue to be widely used worldwide. They stimulate insulin secretion by blocking ATP-sensitive potassium channels in the pancreatic β-cells.

Metformin (a biguanide) is the most widely used oral antidiabetic agent for the treatment of type 2 diabetes. Metformin exerts its glucose-lowering effect by decreasing hepatic glucose output and improving insulin sensitivity. The use of metformin alone is often sufficient to maintain glycemic control but some patients requite a second therapeutic agent in combination.

Most available antidiabetic agents have been used alone and in combination to treat type 2 diabetes. The appropriate choice of agent(s) depends upon the pharmacological properties of the medications and clinical characteristics of the patient. The most commonly used combination therapy is metformin plus a sulfonylurea. Other useful combinations are metformin plus thiazolidinediones (or glitazones), and any of the above agents combined with an α-glucosidase inhibitor, e.g., acarbose, miglitol and voglibose. Other new classes of antidiabetic agents are also beneficially used in various combinations, including the so-called DPP4 inhibitors and sodium glucose co-transporters (SGLTs).

Effervescent oral pharmaceutical formulations are well known alternatives to pills and capsules, but these formulations present many challenges to the development of stable and attractive dosage forms. Since effervescent systems depend on the reaction between an organic acid or any other constituent that creates an acidic environment and sodium carbonate or sodium bicarbonate, releasing carbon dioxide in aqueous solution, these products unfortunately tend to deliver significant amounts of sodium along with the drug. Recent guidance from organizations such as the World Health Organization and the US Food and Drug Administration (FDA) has suggested decreasing sodium intake in general, and particularly in populations susceptible to health risks if sodium intake is elevated.

Since antidiabetic agents such as metformin are administered chronically, the cumulative amount of sodium delivered by conventional effervescent formulations would be unacceptably high for patients with cardiovascular disease or on low sodium diets. However replacing the sodium salts in a typical effervescent couple with potassium salts is not, straightforward given the physic-chemical properties of such potassium salts and their inherent tendency to he meta-stable (high reactivity). Studies on the effects of potassium supplements were reported by Sellmeyer, D. E., Nutritional Influences on Bone Health, pp 109-117 2013 (“The Effect of Alkaline Potassium Salts on Calcium and Bone Metabolism”). Some potassium supplements are delivered from effervescent solutions, but these are specialized formulations that also do not contain other drug components.

In general, pharmaceutical compositions containing potassium salts are less stable than those containing sodium salts and may require special packaging to deal with the corrosiveness of potassium. But in the particular case of developing an effervescent formulation to replace a conventional tablet, there is the issue of bioequivalence. For instance, in NDA 21-575, Merck and Company reported that of four test formulations of effervescent alendronate meant to be bioequivalent to Fosamax tablets, surprisingly, only two of the formulations had drug absorption comparable to the tablets. These data showed that effervescent formulations can be difficult to prepare as a suitable therapeutic delivery form using conventional effervescent systems based on sodium salts. There is even less experience making effervescing systems with only potassium that are meant to be bioequivalent to an antidiabetic tablet.

### SUMMARY OF THE INVENTION

One object of the invention is to provide a stable effervescent, tablet, granule or powder composition free from sodium introduced by the effervescing couple, comprising:
an effective amount of on or more antidiabetic compounds,
an effervescing acid component essentially free of sodium,
an effervescing base component essentially free of sodium,
buffers, to modulate gastric pH after ingestion, essentially free of sodium;
wherein said composition is completely solubilised within 5 minutes without stirring in 3 to 8 fluid ounces of water at between 5-20° C.

Another object of the invention is to provide a method of manufacturing a stable effervescent tablet granule or powder composition free from sodium introduced by the effervescing couple, comprising:
blending in a fluid-bed granulator an effervescing acid component essentially free of sodium, and an effervescing basic component essentially free of sodium, spray-granulated with purified water or a suitable binder solution, and
adding an effective amount of one or more antidiabetic compounds, tableting, the composition to achieve a tablet hardness of 35 to 120 Newtons, wherein said composition is completely solubilised within 5 minutes without stirring in 3 to 8 fluid ounces of water at between 5-20° C.

Another field of the invention is said product not compressed into effervescent tablets but to be filled into suitable stick pack designs.

### DESCRIPTION OF THE FIGURES

FIG. 1 is a flow chart of the manufacturing process.

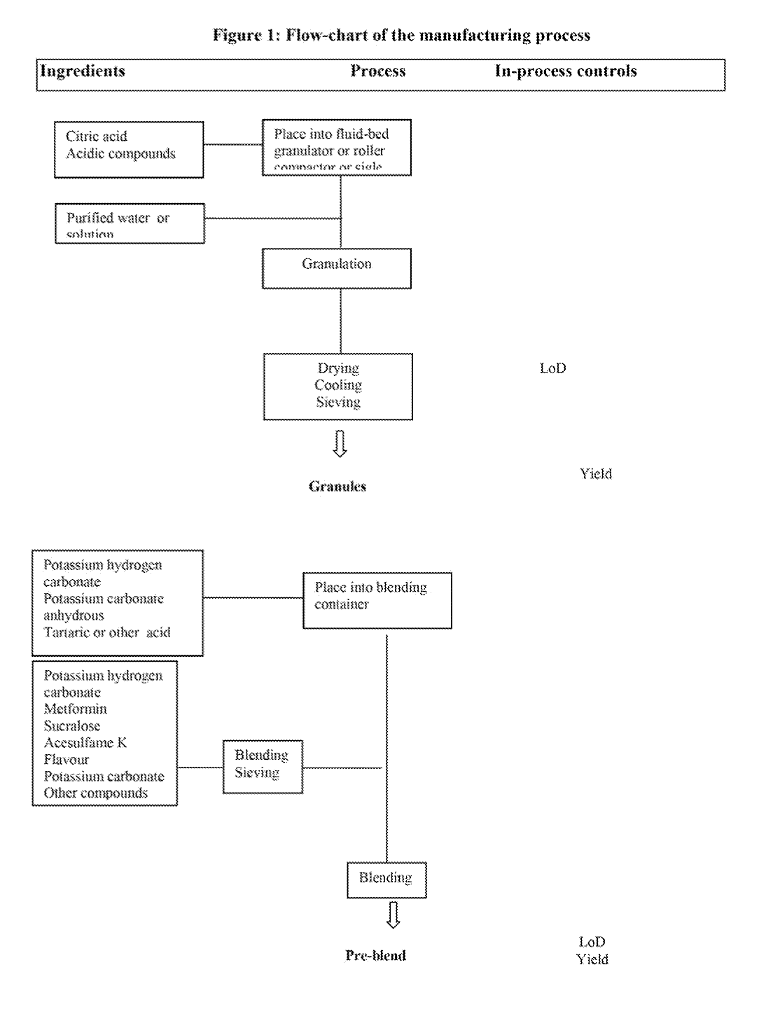

FIG. 2 is a flow chart of the manufacturing process.

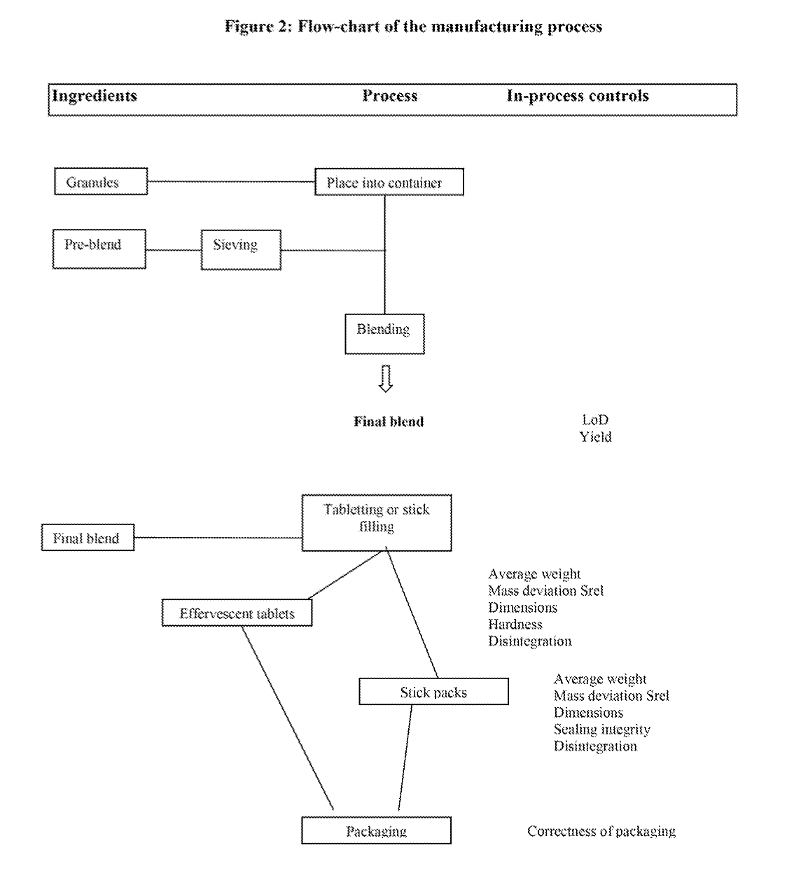

FIG. 3 shows a standard stick pack foil, not containing desiccants of the present invention (hence not the right properties).

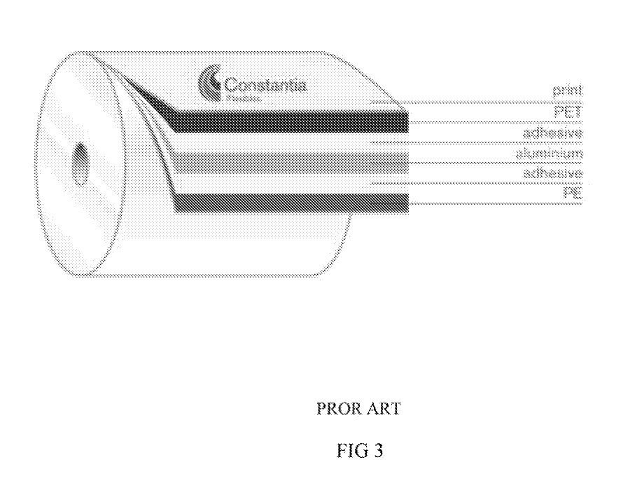

### DETAILED DESCRIPTION OF THE INVENTION

The effervescent system of the present invention is composed of an acid/base couple. The acidic component may be selected from organic acids such as citric acid, tartaric acid, malic acid, fumaric acid, adipic acid, and may include salts of inorganic acids, including dihydrogen phosphate, dipotassium dihydrogen pyrophosphate and potassium acid sulfite and mixtures of the acids, anhydrides and acid salts.

The basic component of the present acid/base couple is a carbonate source selected from potassium bicarbonate, potassium carbonate, potassium sesquicarbonate, potassium glycine carbonate, and mixtures thereof. Minor amounts of sodium salts may be incorporated in the acid/base couple provided that chronic administration of the formula would not deliver more than 50 mg/day of sodium. Buffering components will be selected for compatibility with the effervescent couple components, selected from relevant free acids or salts of the effervescing components and acid/base couples as described above.

Criteria for Obtaining Bioequivalence of Generic Antidiabetic Drugs Using Potassium Salts

Drug products are characterized by and approved on the basis of physiologic responses to administration, such as the pharmacokinetics of drug absorption. New formulations of established drugs must be comparable to already approved versions. Effervescent formulations may, for example, show more rapid absorption and onset o a pharmacodynamic response (an example being acetaminophen and pain relief), or show a very consistent absorption response with respect to the amount of drug absorbed, as with alendronate. Important variables include the physical-chemical properties of the drug itself, particularly water solubility, and the hydrophilic or hydrophobic nature of the drug. The range and extent of drug absorption after oral administration can be further complicated by the physical location of drug absorption within the intestinal tract, as some drugs are only absorbed proximal to the stomach, whereas other drugs may be absorbed through the entire small intestine or in the large bowel. Drugs may be absorbed via specific transport mediated processes, via passive diffusion mediated process, or a combination. Absorption is affected by the physical chemical environment of the gastric milieu, which is influenced by factors such as the presence or absence of food, and especially fats, the rate of gastric transit, and the ionic strength of the gastric and intestinal contents. The present effervescent formulas are designed to match the absorption profile of an immediate release tablet. The ionic strength is elevated to the point where rapid stomach ejection after ingestions is delayed relative to non-ionic solutions or conventional effervescent preparations, but not so elevated that extended stomach retention results. The most direct measure of these parameters is the buffering capacity of the solution, and depending on the administered dose, the ionic, strength is between 2.5 millequivalents and 15 millequivalents of buffering capacity, and preferentially between 4 and 10 millequivalents of buffering capacity.

Consistency of Bio-Absorption

Consistency of absorption from different dosage forms is influenced by many factors such as the disintegration of tablets in the stomach, which can be removed as a variable by delivering a fully solubulized drug in a liquid presentation. In addition to the physical format of the dosing form, consistent absorption is attained if a similar gastric environment is provided during dose administration—such as always administering a dosing form after fasting, or always taking the drug with a meal. An empty stomach is helpful if the drug can be bound or sequestered to components of food. For hydrophobic drugs, however, administering the drug with food or especially a fatty meal may consistently enhance absorption. In one aspect of the present invention, effervescent dosing forms with high ionic strength and high buffering capacity may delay gastric emptying, which can make the absorption parameters more consistent, especially for poorly absorbed drugs. Employing solubulization aids such as emulsifiers also helps to standardize absorption characteristics. Absorption is only one of several factors that can be manipulated by formula components; the rate or speed of absorption is influenced by gastric transit, and the time to maximal concentration in blood is also so influenced.

Special Packaging Due to Potassium

Potassium carbonate and especially bicarbonate have different behaviors compared to their sodium salt equivalents. These are predominantly the following:
Reactivity in the sense of undergoing temperature-induced calcination reaction whilst releasing reaction water. Such water catalyzes the effervescent reaction if used in typical effervescent preparations.
Reactivity in the sense of being sensitive to an acid-base reaction with typical acidic constituents of effervescent preparations, thus forming carbon dioxide which bloats the primary packaging system.
The corrosiveness of potassium salts towards surfaces of aluminum, paper, mineral compounds or other metal-containing compound materials

Taking into account the mentioned properties the selected packaging material needs to fulfill the following criteria: The packaging system of choice contains a water-absorbing polymer, which may be comprised of silica gel-compounds, molecular sieves or the like. It must be pharmaceutically acceptable, attached to the inner wall of the packaging system as discrete platelets or make up a part of the inner sealing layer of the selected packaging foil. Such platelets can be applied online during the packaging process or specifically pre-glued to the foil. The amount of drying capacity should make up for 0.5% of the filling weight of the selected effervescent formulation. Further, the selected packaging system must not contain acidic glues or sealing layers to prevent reaction with the free potassium bicarbonate. Such sealing layers can and even should contain the mentioned desiccant to prevent reaction initiation. Another indispensible property is the ability to absorb water and humidity that protrudes into the system from the ambient surrounding during the storage of the finished product. Such effect is guaranteed by applying opening support only by laser graving that does not affect any layer of the selected packaging system under the aluminum foil. For example, stick packs with such laser cutting enables easier opening but these laser cuts must not be too deep, hence not cut into any layer of the multi-component foil except the aluminum foil. Such aluminum foils are typically 20 mym thick and the depth of the laser engraving is not more than 5 to max. 10 mym.

Potassium salts exhibit an alkaline behavior sufficiently strong to corrode conventional packaging systems not protected with a sealing and adhesive polymer layer that encompasses the said properties.

Removal of all sodium salts from an effervescent couple solves the immediate problem of delivering too much sodium for patients on low sodium diets, but replacing them with potassium salts has an effect on granulation and poses significant challenges with respect to granulation in fluid bed apparatus, granulation in high-shear or single pot apparatus, tableting, disintegration, stability and consumer acceptance. Low or sodium free formulations that maintain high consumer acceptability and tablet or granulate performance characteristics, while producing a stable and acceptable pharmaceutical product, are exemplified above.

The effervescent pharmaceutical formulations of the present invention may be either a tablet or a powder or granule packed off in suitable foils or tubes. To prepare the formulation for ingestion, the tablet or powders are placed in a convenient amount of water, typically 3 to 8 fluid ounces, to produce an effervescent liquid, and the patient drinks the effervescent liquid after reaction has stopped.

In one embodiment the formulation is a tablet, where the total weight of the tablet ranges from about 800 mg to about 5,000 mg. In another embodiment, the tablet weight ranges from about 1500 mg to about 2,000 mg and more particularly from about 3,500 mg to about 6,000 mg.

The amount of active ingredient (API) in the formulation, based on metformin, for example, will range from 50 to 1,500 mg, particularly 100-1000 mg and more particularly about 500 mg of metformin hydrochloride. In some special cases, a dose loading of up to 2,000 mg of individual API components may be incorporated.

In one aspect of this invention the acid source is present in an amount equal to or greater than the carbonate source, on a molar equivalent basis. Thus, when citric acid is the acid source and potassium bicarbonate is the carbonate source, the mole ratio of citric acid/bicarbonate is at least 1:1 to 1:3, for example. An excess of the organic acid, especially citric acid, is preferred because this acid not only efficiently generates the effervescence, but acts as a flavor enhancer. Tartaric acid is taste neutral and allows for innovative flavoring of the citrus fruit standards. In addition to citrus flavouring like lemon, lime, orange and the like, the present invention can incorporate atypical flavourings such as Cola or chocolate, even savoury types such as basil, tomato or meat broth since people will be taking antidiabetic formulations very often (e.g., twice a day) and some variety will be good.

When potassium carbonate is used as the source of carbonate, one equivalent of acid will require a ratio of 2 moles citric acid to 3 moles carbonate. Analogous ratios can be calculated for any source of acid and carbonate, and the carbonate source may be present as a mixture of bicarbonate and carbonate.

For effervescent powder formulations, the composition of the powder is similar to that of the tablet. In preferred formulations the powder is granulated. In one embodiment the effervescing organic acid component contains 20-70% monopotassiwn citrate, preferably 30-60% monopotssium citrate or 40-50% monopotassium citrate.

A preferred composition contains a buffer system of potassium carbonate, potassium bicarbonate and 20-70% mono potassium citrate, resulting in a pH of 4-7 when dissolved in 200 ml of water or a pH of 5-6.

The preferred composition of the invention may have an acid neutralization capacity of 2.5-20 mEq per tablet or 5-16 mEq per tablet.

In another embodiment the effervescent formulation buffers the pH of a patient's stomach for at least 15 minutes, to 30 minutes, or longer.

Conventional formulas use aspartame as a sweetener, which is not ideal due to patients with phenylketonuria issues. Therefore sucralose is a preferred sweetener because it is good tasting and not cariogenic. Acesulfame potassium ideally complements the need for a low-sodium formulation. Stevia derived herbal extracts are another option of choice for the frequent dosing scheme. These sweeteners are selected also for their compatibility (chemical stability) with potassium

The following formulations and manufacturing procedures can be used for manufacture of storage-stable essentially sodium free effervescent tablets or stick pack preparations.

The present method of manufacture should be accomplished with strict adherence to in-process controls. The preferred in-process controls include conventional fluidized bed granulation, which requires the use of an aqueous (or organic) binder solution made from, e.g. PVP (polyvinylpyrrolidone, a water-soluble polymer), HPMC (hydroxypropyl methylcellulose) or sugar alcohols dissolved in water to be sprayed on. The preferred granulation fluid is pure water or a solution of citric acid with the dissolved intense sweeteners in water.

### EXAMPLE 1

| Version | Minimum | | Recommended | | Maximum | |
|---|---|---|---|---|---|---|
| Organic acid | 1000 | | 1500 | | 2000 | |
| Sugar alcohol | 100 | | 199 | | 300 | |
| (Mannitol, | | | | | | |
| Sorbitol) | | | | | | |
| Glidant (Talk, | 0 | | 1.6 | | 2.5 | |
| silica) | | | | | | |
| Metformin HCl | 1000 | | 1000 | | 1000 | |
| Glidant (Talk, | 6 | | 10.5 | | 12 | |
| silica) | | | | | | |
| Potassium | 480 | | 700 | | 850 | |
| carbonate | | | | | | |
| Sucralose | 6 | | 12 | | 20 | |
| Acesulfam K | 15 | | 15 | | | |
| Flavour 1 | 5 | | 5 | | 10 | |
| Flavour 2 | 0 | | 45 | | 100 | |
| Water soluble | 40 | | 51 | | 70 | |
| macrogol | | | | | | |
| Sugar alcohol | 150 | | 180 | | 250 | |
| (Mannitol, | | | | | | |
| Sorbitol) | | | | | | |
| Organic acid | 250 | | 500 | | 750 | |
| Filling/tablet | According to | | According to | | According to | |
| weight | API content | | API content | | API content | |
| Diameter | 20-22 | mm | 22-25 | mm | 25 | mm |
| (tablet) | | | | | | |
| Hight (tablet) | Approx. 5.2 mm | | Approx. 6.0 mm | | Approx. 8 mm | |
| Resistance to | >40 | N | >50 | N | >60 | N |
| crushing | | | | | | |
| Dissolving/ | 1:40 | min:s | 2:00 | min:s | 3:25 | min:s |
| disintegration | | | | | | |
| time | | | | | | |

| | | | |
|---|---|---|---|
| pH-value | Approx. 4.8 | Approx. 4.5 | Approx. 3.9 |
| Odour/Taste | Depends on | Depends on | Depends on |
| | flavour | flavour | flavour |
| Colour of | Nearly clear, | Nearly clear, | Nearly clear, |
| solution | colourless | colourless | colourless |

An appropriate portion of organic acid and potassium carbonate are replaced for non-effervescent ingredients if the stick formulation is applied. Stick formula requires much less effervescence than solid tablets to obtain the same dissolution speed. A specific, powder, granular or crystalline composition that is suitable to be filled into so-called stick-packs. Stick packs are like long and slim sachets (e.g. sugar or instant coffee is marketed that way). A technical summary can be found here: http://www.ropack.com/packaging/stickpacks

Manufacturing Process Development

Effervescent tablet formulations require very low residual humidity levels, extremely low in the presence of potassium carbonate or more pronounced with potassium bicarbonate. Therefore a granulation process followed by a drying step was selected as the basic manufacturing principle.

The residual humidity on a granulate containing potassium bicarbonate should be monitored to be less than 02%.

Finally, the following procedure was identified to manufacture a product that meets the specifications: tartaric acid and a portion of the selected sugar alcohols are pre-blended in fluidized bed equipment and spray granulated with purified water or the solution of citric acid and the intense sweeteners for at least 20 minutes. The resulting granules are dried until the specified loss on drying of <0.15% is achieved (at 75° C., 4 minutes drying duration, 10 g sample).

After a comminution step the granules are blended with the pre-mix. The pre-mix comprises all remaining constituents of the formulation and is manufactured by a series of blending and sieving steps.

Some constituents of the pre-mix require drying in a fluid bed granulator at inlet air humidity levels less than 3 g water/kg air prior to being added to the pre-mix. Alternative drying processes are making the raw materials subject to vacuum at elevated temperatures in a single pot granulator at low shear forces. Typical process parameters are: 40-60° C., a vacuum less than 100-200 mbar. Exposure time should be 60-180 minutes at a mixing rate not exceeding 5 RPM in a 600 L apparatus.

The ready-to-fill or -press mixture is filled into stick packs or compressed into tablets of 18-25 mm diameter and at least 50 N crushing strength on a rotary tablet press, followed by online packaging into strip packs or tubes.

Batch Formula

The production batch size for the manufacture of the commercial good and the clinical medication is 125,000 tablets or around 200,000 stick packs. This number represents the final blend batch size that s compressed into effervescent tablets or filled into stick packs.

Description of Manufacturing Process and Process Controls

The production batch size is 125,000 tablets or 200,000 stick packs. A common granulate comprising most part of the acidic salt or organic or inorganic, acid, or mono potassium citrate and some part of the sugar alcohols is manufactured. Then a pre-mix is manufactured comprising all remaining compounds of the formulation. Finally, the granules and the pre-mix are blended to form the ready-to-press mixture which is compressed into tablets that get strip-sealed in an online process. Alternatively a formula with less effervescent couple is manufactured, if filling of granules into stick packs is the desired finished product.

Preparation of Acidic Granulate (Formulation for 125,000 Tablets or 200,000 Stick Packs):

Citric acid, acidic salts, other organic or inorganic acids, preferably comprising coarse crystals and powder-like qualities, and sugar alcohols are placed into a fluid-bed granulator or single pot granulator and spray-granulated with purified water or a solution of the acidic compound and optionally one ore more intense sweeteners in water. The granules are then dried by vacuum drying or fluidization until a loss on drying of max. 0.15% is achieved. The granules are then cooled down and the loss on drying re-checked. Finally the granules are passed through a 1.5mm sieve and stored in closed container with desiccant. The yield is calculated.

Preparation of Pre-Blend (Formulation for 125,000 Tablets for 200,000 Stick Packs):

A part of potassium hydrogen carbonate, the potassium carbonate anhydrous and remaining ungranulated acidic compound are placed into a container through a sieve of appropriate aperture. Such aperture is selected from 0.8 mm till 1.8 mm., depending on the acidic component used at this step of production. A part of potassium hydrogen carbonate or potassium carbonate, the metformin HCl, sweeteners and flavour are pre-blended for 5-25 minutes and passed through a rotating or oscillating sieve of 0.5-1.5 mm aperture. The remainder of potassium hydrogen carbonate or carbonate is passed through the sieve. The container is blended for 30 min. Finally the loss on drying is tested. The maximal LOD limit has been established at 0.20%. A higher LOD can be tolerated and offset against the determined LOD of the granules. The yield is calculated.

Preparation of Final Blend (Formulation for 125,000tablets or 200,000 Stick Packs):

The acidic granules are placed into a container. The previously prepared pre-blend is then added to the mono potassium citrate or similarly composed granules through typically a 1.5 mm sieve and blended for 30-45 minutes at 5-10 RPM. The loss on drying (max. 0.25%) is checked and the yield calculated. The final blend is packed into PE bags with desiccant and then into steel container for further processing.

Compressing or Stick Filling.

The ready-to-press or to-fill mixture is compressed on a rotary tablet press (Korsch or equivalent) into tablets of 18-25.3 mm diameter, 4.4-7.6 mm thickness with an average mass of 1000-6050 mg, depending on the desired and targeted dosing strength oft the tablet. During compressing the following IPCs are performed:
Appearance
Dimensions
Average mass
Standard deviation
Hardness
Disintegration time

It should be understood that one skilled in this art will recognize equivalent formulations which are intended to be included with the scope of this invention. A very innovative alternative to tablet compression is stick filling. If such process is applied, the following processing steps are executed:

The readymix is loaded onto a stick filling line with 4-10 parallel filling stations (Merz or equivalent). Via a volumetric dosing unit the correct filling weight is adjusted and monitored throughout the filling process. Further in process controls are: length of the foil per stick, sealing pressure, sealing temperature, weight per filled stick.

It can be assumed that such process is economically superior to conventional tableting process,

Controls of Critical Steps and Intermediates

TABLE 4
In-Process controls

| | | |
|---|---|---|
| Test | Limits | Method/Intervals |

Acidic granules

| | | |
|---|---|---|
| Loss on drying | Max. 0.15% | Thermo balance/at the end |
| HR73/75° C./10 g/ | | |
| 4 min | | |
| Yield of final blend | 97.0-100.0% | At the end |
| | of theoretical yield | |

Pre-blend

| | | |
|---|---|---|
| Loss on drying | Max. 0.20% or offset | Thermo balance/at the end |
| HR73/75° C./10 g/ | against the LOD of the | |
| 4 min | granules. | |
| Yield of final blend | 98.0-101.5% | At the end |
| | of theoretical yield | |

Final blend

| | | |
|---|---|---|
| Loss on drying | Max. 0.25% | Thermo balance/at the end |
| HR73/75° C./10 g/ | | |
| 4 min | | |
| Yield of final blend | 97.5-101.0% | At the end |
| | of theoretical yield | |

Tablets

| Diameter | 18.0-25.3 | mm | Calliper, at beginning |
|---|---|---|---|
| Thickness | 4.4-7.6 | mm | Calliper, every 20 min. |
| Resistance to | 50-100 | N | Ph. Eur., current edition, |
| crushing | | | every 20 min |
| Average mass of | 1000-6050 | mg | Ph, Eur., current edition, |

| | | |
|---|---|---|
| tablets | | every 20 min. |
| Uniformity of | RSD max. 3.0% | Ph. Eur., current edition, |
| mass | | every 1 hour |
| Disintegration | Max. 3 min. | Ph. Eur., current edition, |
| | | at the beginning. |

Packaging

| | | |
|---|---|---|
| Correctness of | Has to comply | Visual |
| Lot-No. | | |
| Correctness of | Has to comply | Visual |
| expiry date | | |

Part of the API (e.g., metformin HCl and DPP-4 inhibitors and/or SGLT-2) may be present in a delayed release composition containing a pharmaceutically acceptable salt, hydrate, solvate, polymorph, stereoisomer, ester, prodrug or complex thereof; or optionally may be combined with another API (e.g., metformin hydrochloride mixed with a compound selected from the group consisting of glipizide, glyburide, pioglitazone hydrochloride, repaglinide, rosiglitazone maleate, saxagliptin and sitagliptin phosphate.

A delayed-release component of the API may contain 2-10% by weight of a second effervescing base component (e.g., potassium bicarbonate, potassium carbonate, potassium sesquicarbonae, potassium glycine carbonate, and mixtures thereof), and one or more coatings such as film coating, enteric coating, bioadhesive coating, diffusion coating, and other non-water-permeable coatings known in the art.

These coatings can be functional or non-functional. A functional coating helps slow the release of the active ingredient at the required site of action. In one example, the coating prevents the API from contacting the mouth or esophagus thereby masking its taste. In another example, the coating remains intact until reaching the small intestine (e.g., an enteric coating). Dissolution of a pharmaceutical composition in the mouth can be prevented with a layer or coating of hydrophilic polymers such as cellulose or gelatin. Eudragit® of various grades or other suitable polymers may be incorporated in coating compositions to release the API in the colon.

Coating agents include, but are not limited to, polysaccharides such as maltodextrin, alkyl celluloses such as methyl or ethyl cellulose, hydroxyalkylcelluloses (e.g. hydroxypropylcellulose or hydroxypropylmethylcelluloses); polyvinylpyrrolidone, acacia, corn, sucrose, gelatin, shellac, cellulose acetate pthalate, lipids, synthetic resins, acrylic polymers, polyvinyl alcohol (PVA), copolymers of vinylpyrro and and vinyl acetate (e.g. marketed under the brand name of Plasdone® and polymers based on methacrylic acid such as those marketed under the brand name of Eudragit®.

Excipients can be included along with the film formers to obtain satisfactory coatings. These excipients can include plasticizers such as dibutyl phthalate, triethyl citrate, dibutyl sibacate, triacetine, polyethylene glycol (PEG) and the like, antitacking agents such as talc, stearic acid, magnesium stearate and colloidal silicon dioxide and the like, surfactants such as polysorbates and potassium lauryl sulphate, fillers such as talc, precipitated calcium carbonate, polishing agents such as beeswax, carnauba wax, synthetic chlorinated wax and opacifying agents such as titanium dioxide and the like. All these excipients can be used at levels well known to the persons skilled in the art.

Non-permeable coatings of insoluble polymers, e.g., cellulose acetate, ethylcellulose, can be used as enteric coatings for delayed/modified release by inclusion of soluble pore formers in the coating, e.g., PEG, PVA, sugars, salts, detergents, triethyl citrate, triacetin, etc. The slow release pharmaceutical compositions of the invention can be coated by a wide variety of methods. Suitable methods include compression coating, coating in a fluidized bed or a pan and hot melt (extrusion) coating. Such methods are well known to those skilled in the art. The preferred controlled-release coatings are applied to the API using non-aqueous systems to protect the second effervescing base component from water.

To control the release kinetics pore formers can be added to tall of the above mentioned film formulations. Typical excipients are: Lactose, sugar alcohols like Mannitol, Erythritol, Sorbitol, Lactitol or salts like potassium sulfate or potassium acetate or other equivalent ingredients known to the skilled in the art.

| Examples of combinations | | Weights A/B | Dosages |
|---|---|---|---|
| Caniglaffozin/Metformin | | 100 mg/250 mg, 200 mg/500 | Inital: 100 mg/250 mg once or |
| | Invokana ® | mg, 300 mg/500 mg | twice daily |
| | | 50 mg/850 mg, 150 mg/850 mg, | Range: up to 300/2000 mg |
| | | 50 mg/1000 mg, 150 mg/1000 mg | Dose: Taken once or twice daily |
| Glyburide/Metformin | | 1.25 mg/250 mg, 2.5 mg/500 | Initial: 1.25 mg/250 mg once or |
| | Glucovance ® | mg, 5 mg/500 mg | twice daily |
| | various generics | | Range: up to 20/2000 mg |
| | | | Dose: Taken once or twice daily |
| Glipizide/Metformin | | 2.5 mg/250 mg (pink), | Initial: 2.5 mg/250 mg daily or |
| | Metaglip ® | 2.5 mg/500 mg (white), 5 mg/500 | 2.5 mg/500 mg twice daily |
| | various generics | mg (pink) oval tablets | Range: up to 20/2000 mg |
| | | | Dose: Taken once or twice daily |
| Rosiglitazone/Metformin | | 2 mg/500 mg (pale pink), 2 | Initial: 2 mg/5000 mg once or |
| | Avandamiet ® | mg/1000 mg (yellow), 4 mg/500 | twice daily |
| | various generics | mg (orange) 4 mg/1000 mg | Range: up to 8 mg/2000 mg |
| | | (pink) oval tablets | Dose: Taken twice daily |
| Pioglitazone/Metformin | | 15 mg/500 mg, 15 mg/850 mg | Initial: 15 mg/500 mg or 15 |
| | ActoPlus Met ® | (white to off-white) oblong | mg/850 mg once or twice daily |
| | various generics | tablets | Range: up to 45 mg/2550 mg |
| | | | Dosed once or twice daily |
| Pioglitazone/Glimepiride | | 30 mg/2 mg, 30 mg/4 mg (white | Initial: 30 mg/2 mg or 30 mg/4 |
| | Duetact ® | to off-white) tablets | mg once daily |
| | | | Range: max of one tablet daily |
| | | | Dose: Taken once daily |
| Rosiglitazone/Glimepiride | | 4 mg/1 mg (yellow), 4 mg/2 mg | Initial 4 mg/1 mg or 4 mg/2 mg |
| | Avandryl ® | (orange), 4 mg/4 mg (pink) | once daily |
| | various generics | rounded triangle tablets | Range: up to 8 mg/4 mg |
| | | | Dose: Taken once daily |
| Sitagliptin/Metformin | | 50 mg/500 mg (light pink), 50 | Initial: 50 mg/500 mg or 50 |
| | Janumet | mg/1000 mg | mg/1000 mg twice daily |
| | | (red) oblong tablets | Range: up to 100 mg/2000 mg |
| | | | Doser: Taken twice daily |
| Repaglinide/Metformin | | 1 mg/500 mg (yellow), 2 mg/500 | Initial: 1 mg/500 mg twice daily |
| | PrandiMet ® | mg (pink) tablets | Range: 10 mg/2500 mg, Max per |
| | | | dose |
| | | | 4 mg/1000 mg |
| | | | Dose: Taken twice or three times |
| | | | daily |
| Pioglitazone/Metformin | | 15 mg/1000 mg, 30 mg/1000 | Initial: 15 mg/1000 mg or 30 |
| XR | | mg (white to off-white) round | mg/1000 mg once daily |
| | ActoPlus Met XR ® | tablets | Range: up to 45 mg/2000 mg |
| | | | Dose: Taken once daily |
| Saxagliptin/Metformin | | 5 mg/500 mg (light brown to | Initial: 5 mg/500 mg or 5 |
| XR | | brown), 5 mg/1000 mg (pink), | mg/1000 mg once daily |
| | Kornbiglze XR ® | 2.5 mg/1000 mg (pale yellow to | Range: up to 5 mg/2000 mg |

| | |
|---|---|
| light yellow) capsule-shaped tablets | Dose: Taken once daily |

For fixed dose combinations, although metformin is sometimes dosed once, and more often twice (and sometimes even three times) per day, some of these drugs used in combination (like Januvia, the DPP4 inhibitor sitagliptan, or like the SGLT2 inhibitor canigloflozin), are taken once per day. In such instances a cumulative daily dosing approach is preferred. For example, if the desired dose were 500 mg of metformin twice per day and 100 mg canagiflozin once per day, one could provide a dose of 500 mg metformin plus 50 mg canagliflozin twice per day, which keeps the drug product and dosing regimen simple.

The foregoing examples have been presented for the purpose of illustration and description only. The scope of the invention is to be determined from the claims appended hereto.

1. A stable effervescent tablet, granule or powder composition free from sodium introduced by the effervescing couple, comprising;
an effective amount of one or more antidiabetic compounds;
an effervescing acid component essentially free of sodium,
an effervescing base component essentially free of sodium: and
buffers, to modulate gastric pH after ingestion, essentially free of sodium;
wherein said composition is completely solubilised within 5 minutes without stirring in 3 to 8 fluid ounces of water at between 5-20° C.

2. The composition of claim 1, having tablet hardnesses in the range of 60 to 90 Newtons.

3. The composition of claim 1, having a disintegration time between 60 and 130 seconds.

4. The composition of claim 1, wherein the effervescing organic acid component contains 40-60% potassium salts of pharmaceutically acceptable organic or inorganic acids.

5. The composition of claim 1, further comprising a buffer system of potassium carbonate, potassium bicarbonate and 20-70% acidic component, resulting in a pH of 4-7 when dissolved in 200 ml of water.

6. The composition of claim 5, wherein the buffer system results in a pH of 5-6 when dissolved in 200 ml of water.

7. The composition of claim 5, having an acid neutralization capacity of 10-20 mEq.

8. The compostion of claim 5, which buffers the pH of a patient's stomach for at least 15 minutes.

9. The composition of claim 1, wherein the antidiabetic compound is metformin.

10. The composition of claim 9, further comprising a Canagliflozin.

11. The composition of claim 9, further comprising a Gliptine.

12. The composition of claim 9, further comprising a Glitiozin.

13. A method of manufacturing astable effervescent tablet, granule or powder composition free from sodium introduced by the effervescing couple, comprising:
blending in a fluid-bed granulator or roller compaction or granulation in a single pot apparatus, an effervescing acid component essentially free of sodium, and an effervescing basic component essentially free of sodium, spray-granulated with purified water,
adding an effective amount of one or more antidiabetic compounds, and
compacting the composition and, stick-filling to package, wherein said composition is completely solubilised within 5 minutes without stirring in 3 to 8 fluid ounces of water at between 5-20° C.

14. The method of claim 13, wherein the antidiabetic compounds are metformin and a Canagliflozin.

15. The method of claim 13, wherein the antidiabetic compounds are metformin and a Gliptine.
